(19) United States
(12) Patent Application Publication
(10) Pub. No.: US 2026/0062665 A1
(43) Pub. Date: Mar. 5, 2026

(54) Methods for creating value-added biomass products using natural microbiomes for application as fertilizer enhancer

(71) Applicant: MicroBios S.A., Montevideo (UY)

(72) Inventors: Sven Gross-Selbeck, Vienna (AT); Marcel Burtscher, Itapua (PY); Walter Sandoval, Luque (PY)

(21) Appl. No.: 18/818,942

(22) Filed: Aug. 29, 2024

(51) Int. Cl. C12N 1/20; C05F 17/20; C12N 1/04; C12N 1/14; C12P 39/00

(52) U.S. Cl. CPC C12N 1/20; C05F 17/20; C12N 1/04; C12N 1/14; C12P 39/00

(57) ABSTRACT

The present application provides for a method of creating a synthetic microbiome composition, and the composition. The method comprises a first liquid fermentation phase comprising propagating a microbial consortium in a fermentation solution to generate an inoculum; a second solid fermentation phase comprising applying the inoculum to a solid biomass for a period of time to modify a microbiome of the solid biomass and provide a synthetic microbiome; and processing the synthetic microbiome for application to an environment.

### BACKGROUND OF THE DISCLOSURE

The health of soil depends largely on the microorganisms that colonize this habitat. This microbial community is known as the soil microbiome. Similarly, the optimal development of plants is determined by the quality of the soil, in terms of its physicochemical characteristics as well as its microbial load and diversity. Members of the soil microbiome communicate with plants through phytohormones and other small molecules, establishing a mutually beneficial symbiotic relationship. For example, some bacteria help plants obtain nitrogen from the atmosphere and nutrients from the soil, including metals, phosphates, and others. They can also help plants in situations of physical stress such as drought, or in the fight against pathogens. Taken together, the metabolic versatility conferred by microbes can increase crop yields with relatively less fertilizer input. In view of these characteristics, the use of soil microbes as inoculants has gained traction over the past years, especially in traditional and extensive agriculture. However, in this context, organic farming can take advantage of the knowledge gained about microbes and their benefits.

There are two main approaches for formulating microbial inoculants: i) a reductive approach that focuses on isolating microbes from the complexity of the soil or rhizospheric microbiome, and ii) the formulation of microbial consortia, or even complex microbial communities. The former focuses on specific metabolic traits, whereas the latter takes advantage of the synergies among microbes and their corresponding diverse metabolic capabilities.

Using molecular techniques, such as the sequencing of microbial genes present in the soil, it is possible to study and design microbiomes with the desired metabolic attributes. Likewise, it is feasible to compare these engineered microbial consortia with natural microbiomes of rich and unperturbed soils, such as those from a primary forest, or soil associated with the rhizosphere of healthy and vigorous plants. This can potentially serve as a baseline to define how an optimal soil microbiome is constituted. However, plant-associated microbiomes are dynamic in terms of space (plant anatomy) and time (development phase), meaning that plants will be associated with microbes needed, but only based on those already present in the soil. This underscores the importance of the soil microbiome.

The layer of our planet that contains life, which is known as the biosphere, contains trillions of microbes. The genetic and metabolic diversity conferred by these microorganisms impacts our life. They are responsible for the biogeochemical homeostasis in our planet, which involves nutrient cycling including carbon, sulfur, nitrogen, oxygen, phosphorous, and other elements. Since microbes, including bacteria and archaea, have been around for many years prior to plants and animals, they have set the biochemical stage where multicellular organisms have developed. This points towards the coevolution of microbes with every other organism that evolved afterwards, from fungi, to plants, to animals.

Due to intertwined dependency, several metabolic functions that are required by larger organisms, including plants, are strictly conferred by microbes, such as nitrogen fixation, nutrient acquisition, vitamins synthesis, and others. Therefore, the soil microbiome, or the pool of microbes present in this habitat, determines how rich and dynamic it is, and thus, the extent of metabolic activities that plants can rely upon. The higher the soil microbial diversity, the higher the chances are that plants will develop optimally. Unfortunately, current farming practices, such as tilling, intensive use of agrochemicals, and others, do not protect the soil, and have reduced soil microbiome diversity, which in turn, makes plants require higher fertilizer input, potentially creating a vicious cycle.

Microbial communities are composed of individuals, of the same or different species, or even strains. These individuals, in turn, are part of populations that interact with others, forming communities. Every level has an impact on each other. Therefore, considering genetic diversity, each individual contributes to the function of the ecosystem. Moreover, the interactions can take different forms, depending on the benefit each member acquires. For instance, there is mutualism when both partners benefit, commensalism, when one of them benefits, and a mensalism when one of them inhibits the growth of the other, for instance, by generating antibiotics. It can be a competing relationship when both need the same nutrients. One organism can consume the other, in a predator-prey-type relationship. Synergism occurs when there is greater benefit for both partners.

The study of microbiomes greatly benefited from advances in high-throughput DNA sequencing techniques. Microbiome members can be identified by sequencing the prokaryotic 16S rRNA gene, which functions as a bar code. Other technologies include shotgun metagenomic sequencing, which, in addition to answering the question of “who”, also identifies other genes present in the sample. This allows for the functional determination of the microbiome. The community can also be studied in terms of its diversity, via the calculation of alpha and beta diversity indices. The former describes how diverse a sample is (or community), whereas the latter compares diversity between samples. Typically, the soil and rhizosphere are colonized by bacteria belonging to the phyla Firmicutes, Bacteroidetes, Actinobacteria, and Proteobacteria. The biological and physicochemical conditions determine their relative abundance.

A previous study on different soil types in terms of their microbial cellular abundance and genome complexity revealed that pasture and arable soil contain one order of magnitude higher cell count than forest and even marine soils. However, when looking at the genome complexity, forest and marine soils can be 17 and 32 times more complex, respectively, than arable soil. Pasture soil was equivalent to forest. As genome complexity is required for metabolic diversity, these observations highlight that microbial cell quantity does not necessarily confer quality. Moreover, the fact that pasture soil is more diverse than arable soil might be due to cow dung. Interestingly, manure is a key element in organic fertilization. In that sense, a recent study demonstrated an impact on the soil microbiome by compost application, increasing its diversity, and thus its quality. Overall, these observations suggest that using complex microbial communities on the soil can impact its microbiome, improving its qualities. In other words, they work as soil amendment. Such practices have been traditionally used in organic farming, successfully. This emphasizes the role of microbes impacting the soil microbiome and its properties, which leads to the success of this type of fertilization.

The structure of rhizospheric microbiome depends on the developmental stage of the plant. Thus, as the plant grows, it generates compounds, such as phytohormones, that can be recognized by microbes, promoting their assembly around the root. This dynamic is the foundation for the synergetic interrelation between plants and their root microbiome. Thus, a diverse soil microbiome is needed to supply plants with the key microbes that will sustain their optimal growth.

Biofertilizers are products whose active ingredients are not “chemical” based, but rather of biological origin. However, there are many terms that are applied to this general description, depending on the geographical location and alternatives on the formulations. Some of those terms are bioactivators, biostimulants, phytostimulants, biologics, bioinoculants, bioformulations, bioadditives, etc. However, to consolidate its definition, the European Biostimulants Industry Council (EBIC), defines biostimulants as: “substance(s) and/or microorganisms whose function when applied to plants or the rhizosphere is to stimulate natural processes to enhance/benefit nutrient uptake, nutrient efficiency, tolerance to abiotic stress, and crop quality.” Similarly, the 2018 United States Farm Bill defines bioinoculants as “a substance or microorganism that, when applied to seeds, plants, or on the rhizosphere, stimulates natural processes to enhance or benefit nutrient uptake, nutrient use efficiency, tolerance to abiotic stress, or crop quality and yield.” In addition to the characteristics previously mentioned, others include: i) the increase of beneficial microbes associated with the plants; ii) their capacity to provide non-traditional plant nutrients; and iii) their ability to improve soil quality and health. In the context of organic farming, the fact that biostimulants can increase nutrients uptake indicates that they can act as “bioactivators” of traditional fertilizers. This can help transition large-scale agriculture to a more sustainable path. Similarly, organic farming can get a boost from the use of microorganisms.

When developing biostimulants, there are several characteristics to be followed. For instance, the biostimulants: i) should not be toxic, and must be safe for the environment and animals; ii) should be of natural origin (i.e., isolated from the natural environment); iii) should be bioactive (i.e., being able to interact with plants or intended target); iv) ideally be robust in terms of their compatibility with formulation ingredients; v) must be cost-competitive with the stablished market; vi) must have a positive effect either on the plant (biomass or product yields), and finally, in order to protect producers from technologies of dubious bioactivity, vii) must be evaluated in the field, ideally over multiple seasons.

The active ingredients of biostimulants can comprise numerous biological agents. Some of those can include beneficial bacteria, which are also known as plant-growth promoting bacteria. It can also include beneficial fungi, such as Trichoderma spp. In addition to these whole cells, it can also include microbial byproducts, including humic acids and fulvic acid, seaweed extract, protein hydrolysates, including amino acids and peptides, biopolymers, including chitosan. Some formulations also include inorganic compounds, which are basically trace elements and other nutrients.

Microbial isolates can only rely on the metabolic capabilities conferred by those microbes. Therefore, the advantages conferred to plants, soil, or whatever system is used, are limited. Microbial consortia, by definition, contain more metabolic diversity. However, it's still lower compared to natural complex microbiomes. Furthermore, while members of the consortia might be compatible, they will not necessarily have synergistic relationships. Phycoterra is a type of soil amendment technology that is chemical based. Therefore, it can only increase the abundance of microbes already present in the soil.

Compost and manure contain complex microbiomes. However, their industrial process is not standardized, which can add reproducibility and scalability issues. Furthermore, in neither case have the microbes present been optimized for farming. For instance, during composting, temperature can reach 60 degrees Celsius or more for long periods, which is not compatible with most soil and rhizosphere microbes. Manure, on the other hand, will contain different microbiome profiles depending on the animal's feed, and it may also contain pathogens. In both cases, they are used with dosages of 200-500 kg/hectare, which is not ideal for use as a fertilizer additive, because of their high volume for each hectare.

While advances in microbiome-based technologies are still in their infancy, organic farming has successfully relied on this approach for fertilization technologies, such as manure and compost, as previously mentioned. However, its underlying microbiology has been largely overlooked.

### SUMMARY OF THE DISCLOSURE

The present application relates to a method and composition which focuses on generating microbial diversity that mimics healthy natural environments, in this case, the soil and the rhizosphere.

The method and technology of the present application reproduce a targeted microbiome by selecting the specific inoculum, growth media and culture conditions. In one instance, the rhizosphere's microbiome of healthy plants is reproduced. This is obtained by employing a two-stage fermentation: a primary liquid fermentation with a combination of microbes, followed by a secondary solid-state fermentation of biomass, using the first stage culture as inoculum. This process allows for the establishment of synergistic microbes. The result of this solid fermentation can then be physically modified to tailor to a particular need and use applications.

The microbiome is generated through a two-stage fermentation process. In the first stage, a microbial consortium comprising approximately (by weight) 40% Trichoderma spp. or Bacillus amylofaciens, 30% Bacillus subtilis or Pseudomonas fluorescens or Bacillus megaterium, 15% Lactobacillus casei or Lactococcus lactis, 10% Lactobacillus lactis or Lactobacillus plantarum, and 5% Saccharomyces cerevisiae or Pichia pastoris is propagated in a submerged fermentation process. For this, a 5 to 7% molasses solution is adjusted to a pH of 6.5 by using NaOH or diluted sulfuric acid and inoculated with 20-25 ml per cubic meter substrate of the described consortium. Afterwards, the microbial consortium is propagated at 20 to 25° C. until the pH decreases to 3.5. Once the pH of the fermentation broth has dropped to 3.5, the product of the submerged fermentation process is used as inoculum for solid-state fermentation.

In the subsequent solid-state fermentation, the inoculum produced in the submerged process is applied to the chosen raw biomass material until it reaches a moisture content of 30%. The solid-state fermentation takes place for between 14 and 56 days under anaerobic to microaerophilic conditions at a temperature of 35 to 40° C. If the moisture content falls below 20% during the fermentation process, the solid material can be remoistened by adding more of the inoculum, or water, until a moisture level of 30% is achieved. This helps to maintain conditions for the growth and activity of the beneficial microorganisms in the microbiome. The result of the solid-state fermentation can then be physically modified to meet a particular application.

This synthetic naturalized complex microbiome provides plants the microbial, and therefore enzymatic, diversity for better nutrient acquisition for optimal growth. This allows significantly higher yields with the same chemical fertilizer input, acting as an additive of the latter, by increasing its availability for the plant. As such, the carbon footprint is lowered per ton of crop produced.

In another instance, the microbiome of worm compost, which is generally known as one of the top organic fertilizers, is replicated under specific conditions. This allows the biotransformation of organic material in a more controlled and efficient manner. In both instances, biomass is bio-transformed into a value-added product.

The method of the present application allows for the generation of microbial communities at an industrial scale, which can be called synthetic naturalized microbiomes. The methods of the present application may allow for replicating root microbiome at industrial scale and replicating worm compost microbiome at industrial scale.

The method of the present application also allows for the enhancement of fertilizers using a synthetic naturalized microbiome; the generation of a plant-growth promoting product that is based on a synthetic naturalized microbiome; the replication of the components of worm compost microbiome at industrial scale, such as intestine and leachate; and the reduction of the carbon footprint in agriculture by using a synthetic naturalized microbiome in combination with traditional fertilizers.

In accordance with a first aspect of the present application, a method is provided, the method comprising a first liquid fermentation phase comprising propagating a microbial consortium in a fermentation solution to generate an inoculum; a second solid fermentation phase comprising applying the inoculum to a solid biomass for a period of time to modify a microbiome of the solid biomass and provide a synthetic microbiome; and processing the synthetic microbiome for application to an environment.

In various embodiments of the method of the first aspect of the present application, the microbial consortium may comprise at least 5% (by weight) Trichoderma spp.; at least 5% (by weight) Bacillus subtilis; at least 5% (by weight) Lactobacillus casei; at least 5% (by weight) Lactobacillus lactis; and at least 1% (by weight) Saccharomyces cerevisiae. The microbial consortium may comprise approximately 40% (by weight) Trichoderma spp.; approximately 30% (by weight) Bacillus subtilis; approximately 15% (by weight) Lactobacillus casei; approximately 10% (by weight) Lactobacillus lactis; and approximately 5% (by weight) Saccharomyces cerevisiae. The microbial consortium may also comprise: at least 5% (by weight) Bacillus amyloliquefaciens; at least 5% (by weight) Pseudomonas fluorescens or Bacillus megaterium; at least 5% (by weight) Lactococcus lactis; at least 5% (by weight) Lactobacillus plantarum; and at least 1% (by weight) Pichia pastoris. The microbial consortium can also comprise: approximately 40% (by weight) Bacillus amyloliquefaciens; approximately 30% (by weight) Pseudomonas fluorescens or Bacillus megaterium; approximately 15% (by weight) Lactococcus lactis; approximately 15% (by weight) Lactobacillus plantarum; and approximately 5% (by weight) Pichia pastoris.

In accordance with various embodiments of the method of the first aspect of the present application, the fermentation solution may comprise a 5 to 7% (w/v) molasses solution. The molasses solution may be mixed with NaOH or diluted sulfuric acid until reaching a pH of approximately 6.5.

In accordance with various embodiments of the method of the first aspect of the present application, the first liquid fermentation phase may comprise propagating the microbial consortium at between 20 to 25° C. until the pH of the fermentation solution reaches approximately to 3.5.

In accordance with additional or alternative embodiments of the method of the first aspect of the present application, the second solid fermentation phase comprises applying the inoculum from the first liquid fermentation phase to the solid biomass until a moisture content of between 20 and 40% is reached. The second solid fermentation phases comprises running the second solid fermentation phase for between 14 and 56 days under anaerobic to microaerophilic conditions at a temperature of 35 to 40° C. If during the second solid fermentation phase the moisture content falls below 20%, the solid biomass may be remoistened by adding more of the inoculum, or water, until a moisture level of 30% is achieved.

In accordance with additional or alternative embodiments of the method of the first aspect of the present application, the solid biomass comprises one or more of plant biomass, manure, rumen, or organic residues. The second solid fermentation phase may further comprise, after applying the inoculum to a solid biomass, physically isolating the inoculated solid biomass. The second solid fermentation phase can be performed under aerobic, microaerobic, or anaerobic conditions.

In accordance with various embodiments of the method of the first aspect of the present application, processing the synthetic microbiome may comprises drying the synthetic microbiome and converting the synthetic microbiome into granules or a powder. The microbiomes of the granules or powder can be liquified via liquid extraction using an isotonic physiological buffer to a final concentration of 1× to 100×, and its supplementation with a carbon source such as molasses at 1 to 3% v/v. The liquid microbiome can used as fertilizer additive or coating. The liquid microbiome may be enriched with one or more bacterial isolates with a desired metabolic trait, such as nitrogen fixation, soil nutrient solubilizer, N₂O (nitrous oxide) metabolizers, plant-growth promoters, and others of agricultural value.

In further embodiments of the method, the microbial consortium comprises worm leachate, and the synthetic microbiome replicates worm leachate and worm intestine microbiomes.

### BRIEF DESCRIPTION OF THE DRAWINGS

FIG. 1 shows a process for generating a naturalized synthetic root microbiome according to the present application;

FIG. 2 shows the structure of the microbiome of different samples according to the present application;

FIG. 3 shows a beta diversity analysis (PCoA, Bray-Curtis) based on 16S rRNA gene sequencing data;

FIG. 4 shows a further beta diversity analysis (PCoA, Bray-Curtis) based on 16S rRNA gene sequencing data;

FIG. 5 shows an evaluation of the microbiome-based additive of the present application in the field;

FIG. 6 shows the percentage of variation of crops treated with a fermented humus product of the present application in replacement of 30% of NPK;

FIG. 7 shows the effect of the liquified microbiome on root length of a crop; and

FIG. 8 shows a further beta diversity analysis (PCoA, unweighted Unifrac) based on 16S rRNA gene sequencing data.

### DETAILED DESCRIPTION OF THE DISCLOSURE

In view of the advantages of microbiome-based approaches, the present application describes a technology developed that promotes plant growth while increasing the efficiency of traditional fertilizers. Because each kilogram of synthetic fertilizer is “bioactivated” by this microbial community, less input is needed to achieve the same or higher yields. This leads to a more sustainable agricultural practice, reducing its carbon footprint. While the data presented below focuses on nitrogen, phosphorous, and potassium (“NPK”), organic fertilizers can also be bioactivated by microbes. As mentioned before, this is due to the synergistic interplay among bacteria that helps in nutrient uptake and efficiency.

According to the present application, a microbiome-based bioadditive was developed, focused on desired and optimal microbial capabilities for plants. For example, nitrogen fixation, reduction of nitrification, the presence of microorganisms that promote plant growth and that increase the bioavailability of nutrients, among other traits. To achieve this, fermentation techniques were employed including a defined microbial consortium cultured in bioreactors and a subsequent solid-state fermentation, employing organic materials in each stage. The microbial consortia contained species, and therefore genes, required for the desired metabolic functions. The microbes included bacteria belonging to the phyla Actinobacteria, Firmicutes, and others.

The fermentative process, along with the consortia members, modifies the structure, diversity, and function of the solid substrate microbiome. Once the fermentation is complete, the bioadditive is granulated for easy field implementation or formulated for liquid application.

Existing microbiome-based organic fertilizers include manure, compost, and “fermented” products, or those that were developed by a hypothesis-driven approach, such as the bioadditive described herein. Manure represents the least developed technologically, as it depends on the animal's dung microbiome, and the biotransformation process is carried out in the field under uncontrolled conditions. Compost is a more controlled process as it is performed in a semi-closed system, where new substrate is added overtime. Here, however, the inoculum and the substrate represent the biggest variables. Finally, there are the fermented products, obtained through controlled bioprocesses in bioreactors with pre-established inoculums and substrates. This controlled environment also allows for optimization. Therefore, fermented products are the most advanced among microbiome-based organic fertilizers.

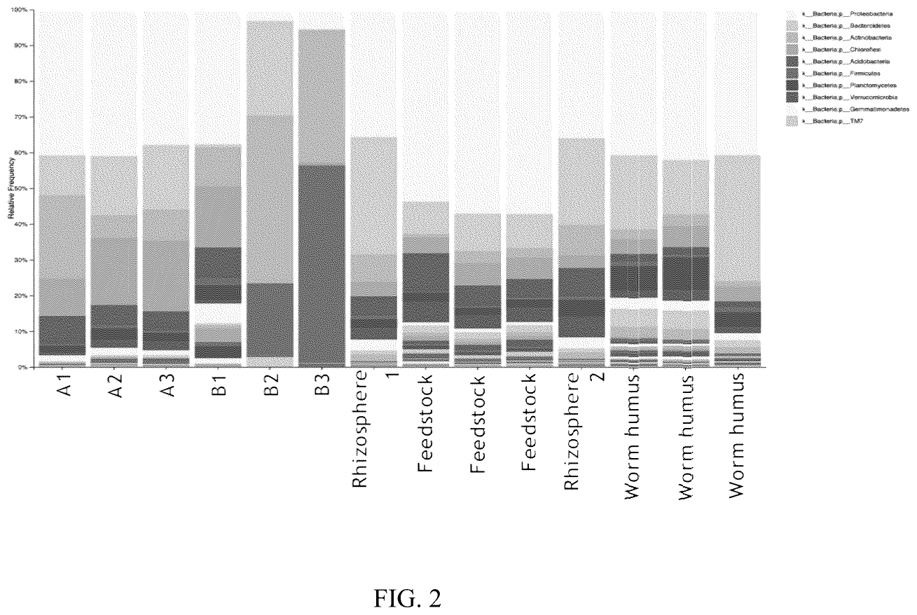

The bioadditive is formulated on a microbiome-based approach where microbial and metabolic diversity are key for its function. Understanding its composition can only be achieved via non-culture techniques such as 16S rRNA sequencing. Thus, metagenomic analyses were performed of the solid substrate, the rhizosphere of healthy and robust plants, the bioadditive as currently used and with variations on its fermentation parameters, and worm humus (FIG. 2).

A first example of a composition according to the present application is a naturalized synthetic rhizosphere microbiome.

The process involves a primary (liquid) fermentation with a microbial consortium, comprising approximately 40% Trichoderma spp., 30% Bacillus subtilis, 15% Lactobacillus casei, 10% Lactobacillus lactis and 5% Saccharomyces cerevisiae, followed by a secondary (solid) fermentation. The liquid fermentation can include a range of each microbe, such as between 5 and 40% Trichoderma spp., between 5 and 30% Bacillus subtilis, between 5 and 15% Lactobacillus casei, between 5 and 10% Lactobacillus lactis and between 1 and 5% Saccharomyces cerevisiae. The percentage amounts can be higher for each than those identified when one strain in the consortium is provided in a lower amount than the identified 40% Trichoderma spp., 30% Bacillus subtilis, 15% Lactobacillus casei, 10% Lactobacillus lactis and 5% Saccharomyces cerevisiae. Thus, the consortium comprises at least 5% of each bacterium and filamentous fungi (Trichoderma) and at least 1% of Saccharomyces cerevisiae. Apart from these microbes, the following can also be used in leu of each individual species at the same proportions as mentioned above: Lactobacillus casei & L. lactis- >Lactococcus lactis, Lactobacillus plantarum; Bacillus subtilis- >Pseudomonas fluorescens, Bacillus megaterium; Trichoderma- >Bacillus amyloliquefaciens; S. cerevisiae- >Pichia pastoris. Furthermore, the whole consortia can be comprised of these alternative microbial species.

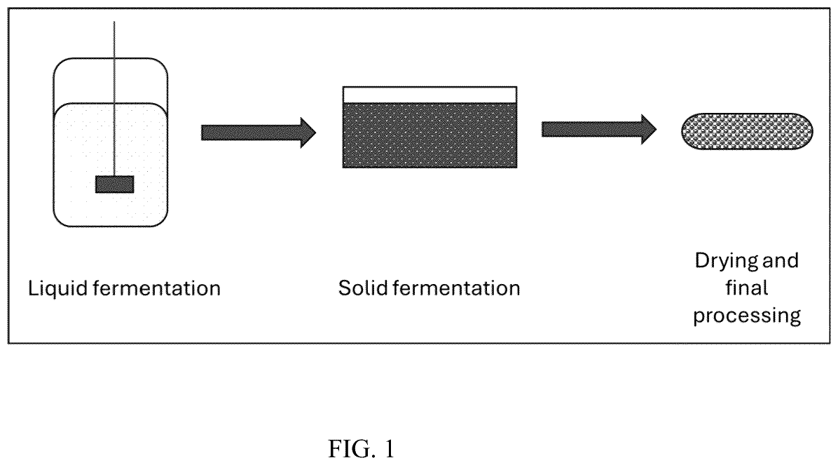

FIG. 1 shows the process for generating a naturalized synthetic root microbiome. A primary liquid fermentation with a defined microbial consortium, followed by a secondary solid-state fermentation using biomass as substrate. Finaly, drying and final processing is performed as appropriate for the end use.

The inoculum comprising a defined microbial consortium is used to ferment molasses (7% w/v) at room temperature (RT). The consortium contains a mixture of 40% Trichoderma sp., 30% Bacillus subtilis, 15% Lactobacillus casei, 10% Lactobacillus lactis, and 5% Saccharomyces cerevisiae. The liquid fermentation can include a range of each microbe, such as 5 to 40% Trichoderma spp., 5 to 30% Bacillus subtilis, 5 to 15% Lactobacillus casei, 5 to 10% Lactobacillus lactis and 1 to 5% Saccharomyces cerevisiae. The fermentation broth is monitored for pH. When this parameter reaches 3.5, the fermentation is complete. Other sugar sources can be used besides molasses, and other residues rich in carbon sources from other industries can be used, such as beet molasses, whey, glycerol, defined sugars such as glucose, fructose, sucrose (or their combination), and/or lactose.

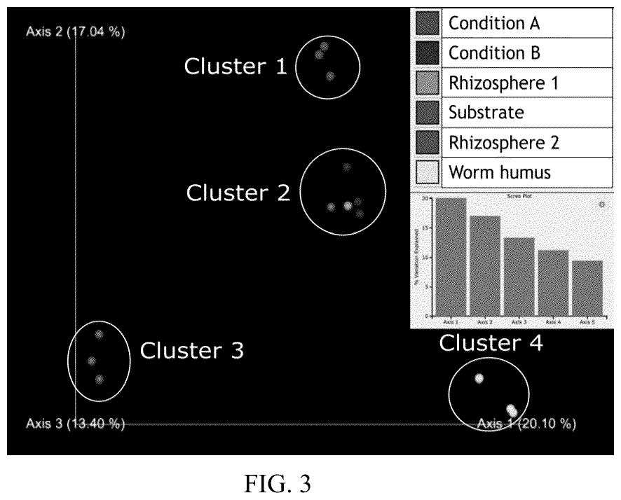

The resulting fermentation is then used to inoculate solid biomass, such as plant biomass, rumen, manure, organic residues, and similar solid biomasses. The inoculation is performed at a ratio such that humidity is approximately 30%. Humidity can also be between 20 to 40%. After inoculation of the solid material, the inoculated solid material is then covered with plastic or any other form of physical isolation. The fermentation is then run for 2 to 6 weeks. This process changes the solid substrate microbiome and transforms it into a microbial community similar to that of the root (FIG. 3, Cluster 2).

After fermentation, the material is processed in a way that is compatible for its field use, such as granulation. It can also be applied in the form of powder. After this, the material is dried by thermal treatment. In addition, the resulting microbiome can be liquified via liquid extraction, and applied along with a carrier that acts as carbon source for the bacteria. For this, the solid microbiome, either immediately after solid-state fermentation or granulation, is resuspended and homogenized in a compatible buffer at 10% w/v. The buffer can be phosphate buffer, saline solution, or any other isotonic physiological buffer. Subsequently, the solids are separated by gravity or centrifugation at 500 rpm for 5 minutes. The supernatant is then centrifuged at maximum speed. The pellet of this centrifugation is then resuspended in fresh buffer at a volume equivalent (1:1) to the initial solid amount, for instance, 100 g into 100 mL. This results in a 1× liquid microbiome. Additionally, this can be further concentrated at 10× and 100× via centrifugation. To this liquefied microbiome, a carbon source can be added, such as molasses at 1.5 to 5% v/v final concentration. This liquified microbiome can be added along with the chemical fertilizers at a dosage of 20 to 50 L per hectare at 1×, 2 to 5 L per hectare at 10×, and 200 to 500 mL per hectare at 100×.

FIG. 2 demonstrates the microbial relative abundance at the phylum level of the solid substrate, the bioadditive generated under two conditions, A: aerobically, and B: microaerobically, and worm humus. The latter is colloquially known as high quality and was used as an external comparison. FIG. 2 shows the structure of the microbiome of the different samples: aerobically (A1, A2, and A3) and anaerobically (i.e., microaerophilic, B1, B2, B3, from different depths) fermented product, natural rhizosphere of vigorous plants (rhizosphere 1 and 2), and earthworm compost, as an external control. A1/A2/A3 were fermented “aerobically” by aerating the solid raw material. On the other hand, B1/B2/B3 were fermented anaerobically (i.e. microaerophilic) by keeping the solids covered, and each sample was taken from different depths. The most optimized product in terms of promoting plant growth, based on field application, corresponds to B3, covered anaerobically fermented raw material. Feedstock samples represent the raw material, or plant biomass, used to perform the solid-state fermentation. Data was generated by 16S rRNA gene sequencing. Additionally, the rhizosphere of healthy and vigorous adult plants was analyzed.

As can be seen, the substrate microbiome can be modified by fermentation conditions, as seen in samples A1/A2/A3, and B1/B2/B3, aerobic and anaerobic (microaerobic) fermentation, respectively. The substrate comprised Proteobacteria as the most abundant phylum. In terms of the bioadditive, the anaerobic method promoted Proteobacteria, whereas in the microaerophilic condition, currently used in the field, Firmicutes was the most abundant. This phylum is comprised of many microaerophilic, facultative, as well as strict anaerobic microorganisms, such as Clostridium sp. Known probiotics such as lactic acid bacteria and bacillus are also members of this phylum. The three anaerobic samples contain different abundance levels of Firmicutes, due to the sampling depth within the solid-state fermentation reactors. For instance, sample B1 looks more like A1, A2 and A3, because it was closer to the surface. On the other hand, worm humus and the rhizosphere contained Bacteroidetes as the most abundant phylum. Overall, this data reveals that the substrate's microbial community can be modified, and the fermentation conditions have a profound effect on the resulting microbiome. As previously mentioned, the bioadditive was submitted to iterative rounds for process development until the current composition was achieved (not shown). Those iterative rounds included changes in the consortia used as inoculum for the liquid stage fermentation; changes in the physicochemical parameters of the liquid and solid-state fermentations; and changes in the final physical treatment and presentation.

Another microbiome analysis performed was beta diversity, which is a comparison of diversities between samples. Similarly, data visualization is carried out via principal coordinate analysis (PCoA), which allows visually clustering samples according to their similarity.

FIG. 3 shows a beta diversity analysis (PCoA, Bray-Curtis) based on 16S rRNA gene sequencing data. Cluster formation indicates that samples are similar in terms of beta diversity. FIG. 3 shows the presence of four clusters among the samples analyzed: aerobic bioadditive (Cluster 1), microaerobic additive with rhizospheres (Cluster 2), the substrate (Cluster 3), and earthworm humus (Cluster 4). This result demonstrates two key points: a) the substrate microbiome composition is modified during the fermentation process, and b) the anaerobic bioadditive has similarities to rhizospheric microbiomes. In other words, the optimized fermentative process and inocula achieve the generation of a naturalized synthetic microbial community.

Therefore, the anaerobic bioadditive provides seeds or seedlings, at the beginning of their growth cycle, the microbiological environment that they would naturally develop under optimal conditions. This allows the plant to “save” metabolic resources and time, allowing it to develop its biomass. In addition, this growth is enhanced due to the high relative abundance of microorganisms that promote plant growth. Another notable point is that the bioadditives are closer to worm humus, compared to the substrate, when analyzed from axis 1.

In addition to comparing the bioadditive with healthy rhizospheres and other samples, a comparison was made of the soil of forests and production fields treated or not with this microbiome-based fertilizer. The treated fields were exposed to the bioadditive for nine consecutive years. The forest and field soils analyzed were in the district of Carlos Antonio Lopez, department of Itapda, Paraguay. The samples were randomly selected, within the first 20 cm from the top, and were not associated with the rhizosphere of any vegetation.

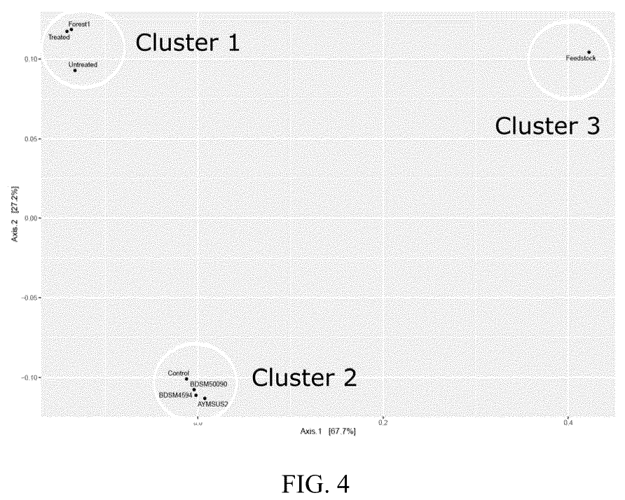

FIG. 4 shows Beta diversity analysis (PCoA, Bray-Curtis) based on 16S rRNA gene sequencing data. FIG. 4 shows three clusters: soil samples (Cluster 1), the bioadditive inoculated with different inocula (Cluster 2), including control (i.e., the bioadditive as commercialized), and the substrate (Cluster 3). Cluster 2 corresponds to different samples of the humus product inoculated with different sets of microbes. Control refers to the actual commercialized product. Treated, untreaded and Forest1 are soil samples from the same location. Treated is soil where the product has been used for 3 years. Untreated is soil where traditional fertilization is performed. Forest1 corresponds to a primary (unperturbed) forest in the area.

Interestingly, while every soil sample is clustered together, the treated soil appears closer to the forest, suggesting a modification of the soil microbiome due to the bioadditive multi-year treatment. While this data represents a single timepoint, it agrees with previous observation with soil amendment practices with compost and longitudinal experiments would reveal the soil microbiome dynamics upon treatment with this microbiome-based additive. However, these results reveal a key point: the bioadditive, in addition to providing plants with a rhizospheric-like microbiome, can potentially modify the soil microbial community to resemble that of the forest. Thus, it suggests the potential of this technology for soil restoration activities, and regenerative agriculture.

Despite the microbial characteristics of a biostimulant, its effectiveness in increasing yields is what finally matters. Therefore, its evaluation is critical. While greenhouse- or lab-scale data are relevant, field applications are fundamental for obtaining robust and reproducible data. This will assure consumers and regulators of the effectiveness of each developed technology. For this purpose, the microbiome-based bioadditive was evaluated in the field by replacing approximately 30% of NPK fertilizer, and comparing it with 100% NPK, under identical conditions.

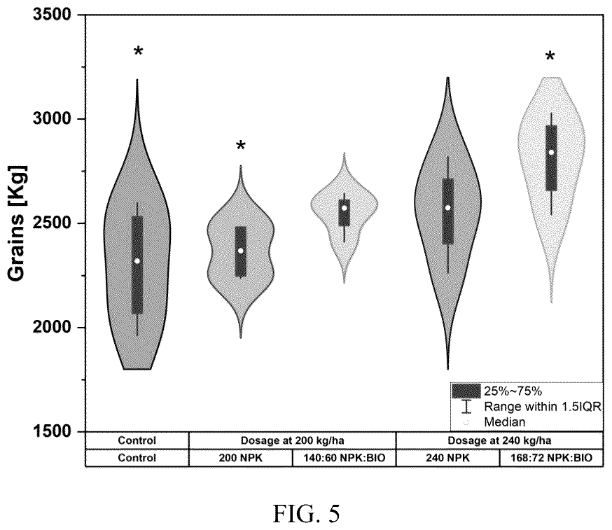

FIG. 5 is the evaluation of the microbiome-based additive in the field by the Paraguayan Institute of Agricultural Technology using soybean (var. SOJAPAR R 19). Assay was performed in Tomas Romero Pereira, Department of Itapda, Paraguay (−26,453196. −55,264015), altitude of 330 meters above sea level (masl), and soil type Rodic Kandiudox, in November 2022 to March 2023. Each treatment was performed with 4 repetitions, each consisting of 9 m long lines. separated by 0.45 m, each. * p<0.05.

As can be seen in FIG. 5, per hectare, 168 kg of bioactivated NPK (4-30-10, proportion) with 72 kg of the bioadditive, increases the yield significantly (ANOVA, p<0.05), compared to negative non-fertilized control (2,812 vs 2,299 kg) and to 200 kg of NPK alone (2,812 vs 2,364 kg). Furthermore, while not significant (p>0.05), this condition produced more than that of 240 kg of NPK alone (2,812 vs 2,557 kg). This field assay was performed in collaboration with the Paraguayan Institute of Agricultural Technology (IPTA, in Spanish).

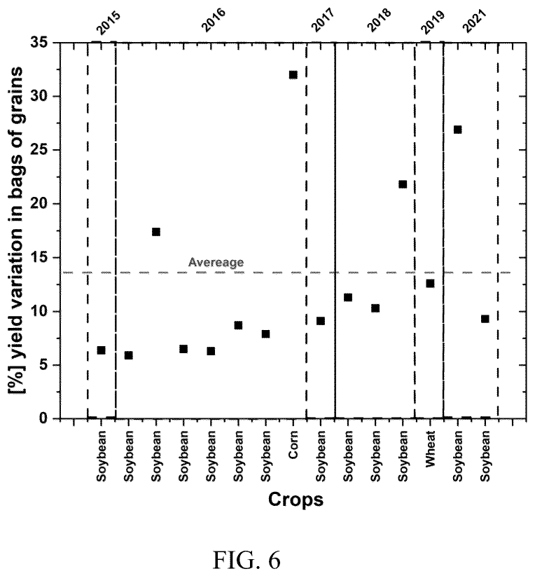

In addition to this field test, production data was analyzed from producers who have tested this microbial biotechnology over the years. FIG. 6 shows the percentage yield variation among different crops from 2015 to 2021, across an applied area of over 2,000,000 acres. These values were calculated considering the treatment (30% bioadditive: 70% NPK) versus control (100% NPK), under the same conditions, side by side. As can be seen, the average percentage yield increase was 12.8%, across multiple years and phylogenetically distinct crops, i.e., legumes and grasses. This confirms that the use of this microbiome-based bioadditive, in tandem with NPK, has a positive effect on crop yield, at large scale.

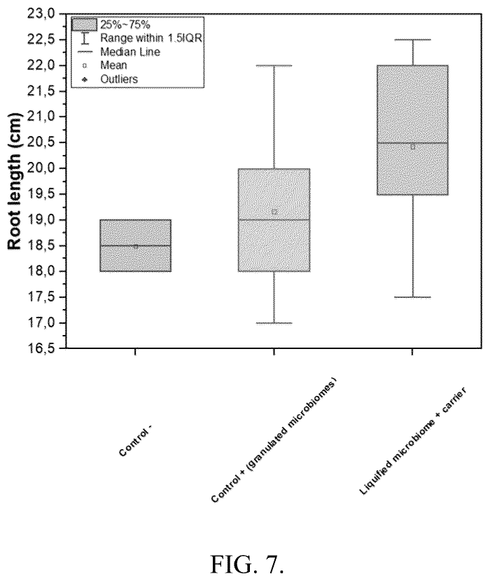

A liquified microbiome from the granulated version was also evaluated (FIG. 7), revealing a positive impact on the root by increasing its length by 10.2% compared to negative control. The liquid microbiome was extracted in physiological buffer at 10% w/v after homogenization and was subsequently concentrated to 1× via centrifugation. The liquid microbiome was supplemented with molasses at 1.5% v/v as carbon and energy source for the microbes. The root length was evaluated after 2 weeks of plant growth (Phaseolus vulgaris, beans).

Considering the microbial diversity within this bioadditive, potential mechanisms of action might involve increase nutrient bioavailability, production of growth-promoting phytohormones, stress tolerance, among others. In this way, production is maximized, while lowering the requirements for chemical fertilizers.

A second example of a composition according to the present application is a synthetic worm compost microbiome.

For this, worm leachate and worm intestine microbiomes can be replicated synthetically, based on beta diversity analysis. In other words, in certain conditions, the resulting microbiome is not significantly different (p>0.05) from the inoculum. For instance, worm leachate can be replicated when used as inoculum (10% v/v) to ferment molasses (7% w/v), for no more than 1 day. For this, a 10% inoculum was used, i.e., 10% of the total volume of the culture. Additionally, worm intestines microbiomes can be replicated either in molasses (7% w/v) or glycerol (10% v/v). For molasses, fermentation can be carried out for 24 hours. In glycerol, fermentation can be carried out for up to 12 days. All fermentations were performed at room temperature. The carbon sources mentioned above were supplemented with nitrogen sources such as yeast extract (2 g/L), ammonium salts (3 g/L), in addition to trace elements, phosphate buffer.

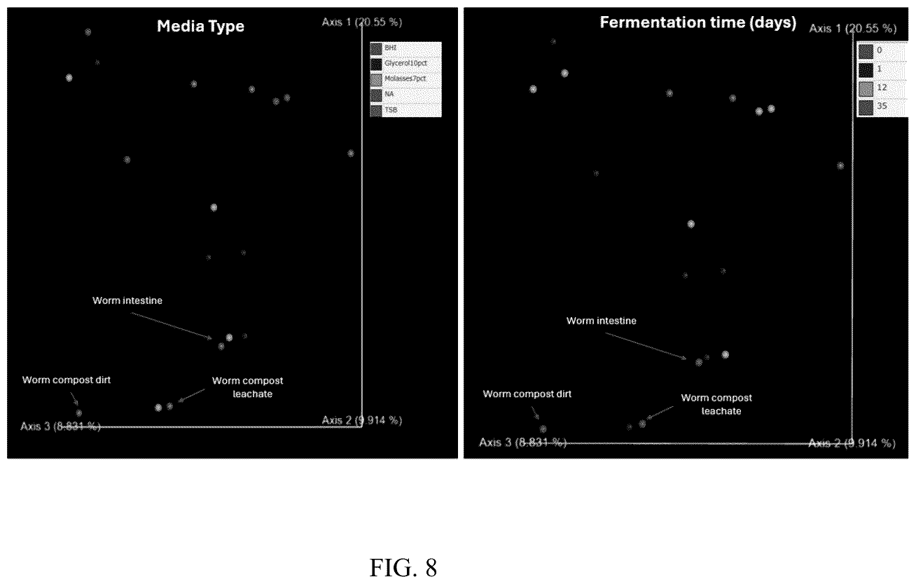

FIG. 8 is the Beta diversity analysis (PCoA, unweighted Unifrac) based on 16S rRNA gene sequencing data. Different media composition and time of fermentation were evaluated using different components of the worm compost as inoculum. Worm compost leachate and worm intestines fermented in molasses (7% w/v), and molasses (7% w/v) or glycerol (10% v/v), respectively, generated microbiomes similar to the initial inocula. The optimal fermentation time for worm leachate in molasses was 1 day, and for worm intestines either in molasses or glycerol was 1 to 12 days. Similar samples cluster.

Table 1 is a statistical comparison (Kruskal-Wallis pairwise comparison) between the results of each fermentation condition (raw glycerol 10%, molasses 7%, and tryptic soy broth (TSB) and the inoculum, in this case worm humus (NA). P-values lower than 0.05 between NA and media types indicate that the resulting microbiomes are significantly different. Therefore, values >0.05 indicates that the resulting microbiome after fermentation were not significantly different from the inoculum. Thus, indicating their similarity.

TABLE 1

| | | Sample | Permu- | | |
|---|---|---|---|---|---|
| Group 1 | Group 2 | size | tations | pseudo-F | p-value |
| BHI | Glycerol10pct | 8 | 999 | 1.473194 | 0.113 |
| | Molasses7pct | 8 | 999 | 1.461141 | 0.115 |
| | NA | 7 | 999 | 2.600635 | 0.027 |
| | TSB | 7 | 999 | 1.043498 | 0.465 |
| Glycerol10pct | Molasses7pct | 8 | 999 | 0.864556 | 0.711 |
| | NA | 7 | 999 | 1.824540 | 0.025 |
| | TSB | 7 | 999 | 1.590421 | 0.091 |
| Molasses7pct | NA | 7 | 999 | 1.402965 | 0.113 |
| | TSB | 7 | 999 | 1.599270 | 0.052 |
| NA | TSB | 6 | 999 | 2.230893 | 0.101 |

Possible applications of the compositions of the present application include: enhancers of fertilizers; plant-growth promoters; soil nutrient solubilizer; increasing fertilizers nutrients availability; biocatalyst of plant biomass for generating other organic value-added products; composting enhancer; soil amendment; use in methods for reducing the carbon footprint of agriculture; and use in methods for reducing the carbon footprint of compost.

Taken together, these data demonstrate that by studying the soil microbiome and its interaction with plants, it is possible to isolate and identify plant-growth promoting microbes. Moreover, their development through microbial biotechnology techniques can lead to the creation of important tools for increasing agricultural yields while achieving its decarbonization. Likewise, organic farming has traditionally relied on microbiome-based fertilization solutions, achieving great and sustainable results. However, with the advent of molecular and fermentation technologies, new and improved solutions can be developed inspired by nature, such us the bioadditive described herein. In this sense, this biotechnology can also be applied in organic farming as a tool to bioactivate their compatible fertilizers. Importantly, however, research and development must be accompanied by field tests over multiple seasons and crops, to safeguard producers. Many questions and challenges remain that academia, government, and industry can tackle together in the pursuit of sustainable food production. Some of those questions include the evaluation of bioactivity of these technologies across geographies, or their exact mechanism of action. Deciphering the soil microbiome is another frontier to be pushed. In conclusion, the strategy to activate fertilizers with next-generation microbiome-based bioactivators represents a great opportunity to achieve greenhouse gas reduction objectives while boosting agricultural production.

What is claimed:

1. A method comprising:
a first liquid fermentation phase comprising propagating a microbial consortium in a fermentation solution to generate an inoculum;
a second solid fermentation phase comprising applying the inoculum to a solid biomass for a period of time to modify a microbiome of the solid biomass and provide a synthetic microbiome; and
processing the synthetic microbiome for application to an environment.

2. The method of claim 1, wherein the microbial consortium comprises:
at least 5% (by weight) Trichoderma spp.;
at least 5% (by weight) Bacillus subtilis;
at least 5% (by weight) Lactobacillus casei;
at least 5% (by weight) Lactobacillus lactis; and
at least 1% (by weight) Saccharomyces cerevisiae.

3. The method of claim 1, wherein the microbial consortium comprises:
approximately 40% (by weight) Trichoderma spp.;
approximately 30% (by weight) Bacillus subtilis;
approximately 15% (by weight) Lactobacillus casei;
approximately 10% (by weight) Lactobacillus lactis; and
approximately 5% (by weight) Saccharomyces cerevisiae.

4. The method of claim 1, wherein the microbial consortium comprises:
at least 5% (by weight) Bacillus amyloliquefaciens;
at least 5% (by weight) Pseudomonas fluorescens or Bacillus megaterium;
at least 5% (by weight) Lactococcus lactis;
at least 5% (by weight)Lactobacillus plantarum; and
at least 1% (by weight) Pichia pastoris.

5. The method of claim 1, wherein the microbial consortium can also be:
approximately 40% (by weight) Bacillus amyloliquefaciens;
approximately 30% (by weight) Pseudomonas fluorescens or Bacillus megaterium;
approximately 15% (by weight) Lactococcus lactis;
approximately 15% (by weight) Lactobacillus plantarum; and
approximately 5% (by weight) Pichia pastoris.

6. The method of claim 1, wherein the fermentation solution comprises a 5 to 7% (w/v) molasses solution.

7. The method of claim 6, wherein the molasses solution is mixed with NaOH or diluted sulfuric acid until reaching a pH of approximately 6.5.

8. The method of claim 1, wherein the first liquid fermentation phase comprises propagating the microbial consortium at between 20 to 25° C. until the pH of the fermentation solution reaches approximately to 3.5.

9. The method of claim 1, wherein the second solid fermentation phase comprises applying the inoculum from the first liquid fermentation phase to the solid biomass until a moisture content of between 20 and 40% is reached.

10. The method of claim 7, wherein the second solid fermentation phase comprises running the second solid fermentation phase for between 14 and 56 days under anaerobic to microaerophilic conditions at a temperature of 35 to 40° C.

11. The method of claim 8, wherein if during the second solid fermentation phase a moisture content of the solid biomass falls below 20%, the solid biomass is remoistened by adding more of the inoculum, or water, until a moisture level of 30% is achieved.

12. The method of claim 1, wherein the solid biomass comprises one or more of plant biomass, manure, rumen, or organic residues.

13. The method of claim 1, wherein the second solid fermentation phase further comprises, after applying the inoculum to a solid biomass, physically isolating the inoculated solid biomass.

14. The method of claim 1, wherein the second solid fermentation phase is performed under aerobic conditions.

15. The method of claim 1, wherein the second solid fermentation phase is performed under microaerobic or anaerobic conditions.

16. The method of claim 1, wherein processing the synthetic microbiome comprises drying the synthetic microbiome and converting the synthetic microbiome into granules or a powder.

17. The method of claim 16, wherein the microbiome of the granules or powder are liquified via liquid extraction using an isotonic physiological buffer to a final concentration of 1× to 100×, and its supplementation with a carbon source such as molasses at 1 to 3% v/v.

18. The method of claim 17, wherein the liquid microbiome is used as fertilizer additive or coating.

19. The method of claim 15, wherein the liquid microbiome is enriched with one or more bacterial isolates with a desired metabolic trait, such as nitrogen fixation, soil nutrient solubilizer, N₂O (nitrous oxide) metabolizers, plant-growth promoters, and others of agricultural value.

20. The method of claim 1, wherein the microbial consortium comprises worm leachate, and the synthetic microbiome replicates worm leachate and worm intestine microbiomes.
